(19) United States
(12) Patent Application Publication
(10) Pub. No.: US 2017/0266347 A1
(43) Pub. Date: Sep. 21, 2017

(54) BIOMIMETIC GRAFT OR IMPLANT AND METHODS FOR PRODUCING AND USING THE SAME

(71) Applicant: The Board of Trustees of the University of Illinoi, Urbana, IL (US)

(72) Inventors: Sriram Ravindran, Chicago, IL (US); Praveen Gajendrareddy, Elk Grove Village, IL (US); Anne George, Chicago, IL (US)

(73) Assignee: The Board of Trustees of the University of Illinois, Urbana, IL (US)

(21) Appl. No.: 15/309,948

(22) PCT Filed: May 13, 2015
(86) PCT No.: PCT/US2015/030466

§ 371 Date: Nov. 9, 2016

Related U.S. Application Data
(60) Provisional application No. 61/992,351, filed on May 13, 2014.

(51) Int. Cl. A61L 27/36; A61L 27/10; A61C 8/00; A61C 8/02; A61F 2/30; A61L 27/06; C12N 5/0775

(52) U.S. Cl. CPC A61L 27/3633; A61L 27/06; A61L 27/10; A61L 27/3608; A61L 27/3683; C12N 5/0662; A61C 8/0006; A61F 2/30767; A61C 8/0013; A61L 2430/02; A61L 2420/02; A61L 2430/12; C12N 2533/00; A61F 2002/3093; A61F 2310/00976

(57) ABSTRACT

Biomimetic grafts or implants coated with an osteogenic extracellular matrix and methods for production and use are described.

### INTRODUCTION

U.S. Provisional Application No. 61/992,351, filed May 13, 2014, the content of which is incorporated herein by reference in its entirety.

### BACKGROUND

In dental and other hard bone implants, osseointegration is the direct structural and functional connection between ordered, living bone and the surface of a load-carrying implant (Branemark (1983) J. Prosthetic Dentistry 50:399-410). Titanium, which is used in many implants, cannot directly bond to living bone or other tissues. Therefore, the process of osseointegration may involve surface modification of titanium implants.

Surface modification methods have focused on increasing surface roughness or creating bioactive surfaces. Surface roughness provides a larger contact area between the metal implant and eventual bone cells. Bioactive surfaces have some effect on protein adsorption, but are more closely related to later adhesion of cells as well as bioactive elements, which could speed up the biological processes associated with wound healing.

The first generation of dental implants were machined (Branemark (1969) Scand. J. of Plast. Reconstr. Surg. 3:81-100) and exhibited reasonable osseointegration characteristics, but suffered from the tendency of the biomechanical stability to decrease over the first few weeks due to bone remodeling. During this process of bone remodeling, the bone necrosis is removed (Branemark (1997) Biomaterials 18:969-978). This process is typically complete in 4 weeks, and then the stability of the implant increases steadily over the subsequent 16 weeks. Despite this 4 month healing time, machined implants have greater than 85% bone to implant contact, and successfully placed implants may last over a decade (Adell (1981) Int. J. Oral Surg. 10:387-416; Branemark (1977) Scand. J. Plast. Reconstr. Surg. Suppl 16:1-132).

The second generation of dental implants sought to modify the implant surface, and a wide variety of implant surface treatment strategies were developed. However, a fundamental understanding of the mechanisms of osseointegration and the specific ways in which surface treatments can accelerate osseointegration is incomplete. The second generation of implants used several surface modification strategies, including media blasting, acid etching, the combination of media blasting and acid etching, controlled oxidation or anodization, laser micro- and nano-texturing, and coatings of calcium phosphate, such as hydroxyapatite. Media blasting creates a randomized, rougher surface with both an increase in average surface roughness as measured by average, peak height as well as a potentially greater peak to valley height of the surface features. Occasionally, particles of the blasting media may be embedded into the surface. Acid etching preferentially attacks grain boundaries, secondary phase particles, or any other site where there is a microstructural or surface energy inhomogeneity. There appears to be minimal effect from acid etching alone in the 0-2 week timeframe after implantation (Celletti, et al. (2006) J. Long Term Effects Med. Implants 16:131-143). However, acid etching after media blasting appears to remove residues and embedded particles from the blasting process, leaving behind a cleaner surface.

The third generation dental implants added further surface treatments in an effort to achieve shorter healing times and better osseointegration. One additional treatment has used storage of blasted and etched implants in dry nitrogen or sterilized saline solution to eliminate carbon contamination and improve hydrophilicity (Rupp, et al. (2006) J. Biomed. Mater. Res. A. 76:323-334). Another such technique involves the creation of a biocompatible titanium hydride layer immediately on the surface of the titanium oxide (Conforto, et al. (2004) Phil. Mag. 84:631-645). Other techniques of “activating” blasted and etched implants include treatment with anions, fluoride treatments, or etching in hydrofluoric acid (Cooper, et al. (2006) Biomaterials 27:926-936). Through such combined mechanical and chemical processing, there have been observed improvements in osseointegration earlier in time, and significant improvements in the 6-12 week timeframe have been observed (Buser, et al. (2004) J. Dent. Res. 83: 529-533; Schwarz, et al. (2007) J. Clin. Periodontol. 34:78-86). Some anodized implants are characterized by a partially crystalline layer enriched in various other ionic species and with an open surface pore structure in the 1-10 micron range. The structural and chemical properties can be altered by changing the anode potential, electrolyte composition, temperature, current, and type of ionic species transported in the solution (Hall & Lausma (2000) Appl. Osseointegration Res. 1:5-8; Frojd, et al. (2008) Int. J. Oral Maxillofac. Surg. 37:561-566). In particular, phosphorous-containing anodized coatings have been shown to promote the early molecular events leading to osseointegration (Omar, et al. (2010) J. Mater. Sci. Mater. Med. 21:969-980). Laser micromachining has also been used to impart both micro-scale and nano-scale texture to an implant surface. The nano-structured surfaces appear to increase long-term interface strength through a coalescence between mineralized bone and the nano-textured surface features (Palmquist, et al. (2010) J. Biomed. Mater. Res. A 92:1476-1486) as well as increasing nearer-term removal torque.

Various coatings have also been applied to implants. For example, plasma spray coatings of metal or calcium phosphate can improve interfacial strength (Cook, et al. (1987) Int. J. Oral Maxillofac. Implants. 2:15-22; Carr, et al. (1995) Int. J. Oral Maxillofac. Implant. 10:167-174). Sputter coatings are dense and uniform, but the process is slower than plasma spray and produces amorphous coatings which may then require subsequent heat treatment to recrystallize. Sputter coating may increase the short time fixation of the implant (3 weeks) but that at longer times (12 weeks) the difference between such coated implants and uncoated ones is negligible (Ong, et al. (2002) J. Biomed. Mater. Res. 59:184-190.). Biomimetic precipitation coatings seek to create calcium phosphate coatings using precipitation from a simulated biological fluid. In one study, in vivo osseointegration was compared for a variety of surface treatments, including uncoated titanium, plasma-sprayed hydroxyapatite, and biomimetically applied hydroxyapatite, all of which were statically indistinguishable (Vidigal, et al. (2009) Clin. Oral Implants Res. 20:1272-7).

U.S. Pat. No. 5,478,327 further discloses an implant coated with a layer of hydroxyapatite. Similarly, WO 02/078759 describes an implant having a layer of a porous metal oxide including amorphous and nanocrystalline calcium phosphate and hydroxyapatite. WO 02/085250 teaches an implant, wherein a coating of resorbable calcium phosphate phases contains adhesion and signal proteins such as bone sialoprotein (BSP), bone morphogenic protein (BMP), fibronectin, osteopontin (OPN), ICAM-I, VCAM and derivatives thereof. Further grafts and implants of this type are described in EP 1166804 and WO 99/08730.

DE 10037850 and WO 03/059407 describe grafts and implants treated with ubiquitin or transforming growth factor (TGF) or systemic hormones such as osteostatin, osteogenic and osteogrowth peptide (OGP). U.S. Pat. No. 7,229,545 teaches bone-analogous coatings made of a collagen matrix mineralized with calcium phosphate. EP 1442755 describes a bioactive ceramic coating composed of osteogenic proteins OP-1, BMP-7 and non-collagenous bone matrix proteins. Osteogenic activities have further been reported for fibroblast growth factor (FGF), transforming growth factor-β (TGF-β), platelet-derived growth factor (PDGF), insulin growth factor (IGF) and family members of the foregoing.

WO 2005/104988 teaches implants and bone repair matrices treated with an under-glycosylated human rBSP. The implants are made of titanium, zirconium, ceramic, metal alloys or stainless steel and may be coated with amorphous or crystalline hydroxyapatite and/or calcium phosphate. Such bone-mimetic coatings however suffer from the disadvantage that they tend to loosen from the substrate with time which affects the long-term stability of implants.

WO 2003/047646 teaches bone grafts that can be fashioned into medical implants. The graft or implant is made of a base material composed of matrix of resorbable polymers or copolymers, and N-methyl-2-pyrrolidone (NMP).

There remains a need to develop treatments of medical implants to promote more rapid and more reliable osseointegration.

### SUMMARY OF THE INVENTION

This invention is a biomimetic graft or implant coated with an osteogenic extracellular matrix. In some embodiments, the biomimetic graft or implant is a surgical implant or dental implant. In other embodiments, the graft or implant is composed of titanium, ceramic or demineralized bone matrix. A kit containing the biomimetic graft or implant coated with an osteogenic extracellular matrix is also provided.

This invention further provides a method for producing a biomimetic graft or implant. This method includes the steps of incubating a graft or implant with undifferentiated mesenchymal stem cells in a first culture medium for sufficient time to promote adhesion of the mesenchymal stem cells to the graft or implant; replacing the first culture medium with an osteogenic culture medium; incubating the graft or implant in the osteogenic culture medium to induce osteogenic differentiation of the mesenchymal stem cells; and subjecting the graft or implant to decellularization thereby producing a biomimetic graft or implant coated with an osteogenic extracellular matrix. In one embodiment, the graft or implant is a surgical implant or dental implant. In another embodiment, the graft or implant is composed of titanium, ceramic or demineralized bone matrix. A biomimetic graft or implant produced by the method is also provided.

This invention also provides a method for promoting in vivo osseointegration or osteoinduction of a graft or implant by implanting a biomimetic graft or implant coated with an osteogenic extracellular matrix in a subject at a site wherein bone tissue and the biomimetic graft or implant are maintained at least partially in contact for a time sufficient to permit enhanced bone tissue growth between the tissue at the site and the biomimetic graft or implant.

### DETAILED DESCRIPTION OF THE INVENTION

A method for enhancing the osteoinduction and/or osteointegration of clinical osteogenic grafts, such as demineralized bone matrices and implant surfaces has now been developed. In accordance with the present invention, the surfaces of grafts and implants are biomimetically enhanced with the native extracellular matrix (ECM) of osteogenic cells prior to implantation into a target implant site. In certain embodiments, the graft or implant surface includes physiologically relevant amounts of growth factors, hydroxyapatite nucleating proteins and other structural proteins that promote host stem cell migration and differentiation and ultimately lead to formation of bone matrices or better osteointegration with the existing bone marrow in the case of implant materials.

Accordingly, the present invention provides a biomimetic graft or implant and method for producing and using the same to promote in vivo osteoinduction and/or osseointegration, wherein the surface of said graft or implant has been modified or coated with the native ECM of osteogenic cells. “Graft” (or “implant”), as used herein, refers to any material, the implantation of which into a human or an animal is considered to be beneficial. An implant may be synthetic (e.g., metal, ceramic, collagen composite, or composite cement) or be obtained from autograft, allograft, or xenograft tissue or combinations thereof, and in the case of mineralized tissues, such as in a bone implant, the implant may include mineralized tissue, partially demineralized tissue, completely demineralized tissue, and combinations thereof. In particular embodiments, the graft or implant of the invention is composed of a demineralized bone matrix.

In some embodiments, the implant is a surgical implant that interfaces with bone when implanted in the patient, such as dental implants, joint replacements (e.g., hip, knee and other joint replacements inserted at one or more points into bone tissue), prostheses inserted into bone, and various types of surgical hardware such as screws, rods, or plates (e.g., for facial or skull reconstruction) that are designed for insertion into bone. Surgical implants refer to those implants that penetrate into the bone (e.g., bone screws), those that may only be found on the surface of the bone (e.g., bone plates, such as those used in assisting fracture healing) as well as those that bone grows into and replaces over time (such as demineralized bone matrix or collagen-based implants, e.g., the INFUSE® Bone Graft).

The surgical implant can be of homogeneous construction, i.e., composed of one type of material, either pure or an alloy or composite, or of heterogeneous construction, i.e., composed of different parts or sections having different types of materials. The surgical implant can include or essentially consist of a metal material, such as titanium, titanium oxide, alloys including titanium, zirconium, zirconium oxide, alloys including zirconium, aluminum, aluminum oxide, alloys containing aluminum, cobalt-chromium alloys, any of the 300 series stainless steels, or any of the 400 series stainless steels. Surgical implants can also be composed of calcium-phosphate-ceramics, bioglass, glass-ceramics, calcium-carbonate, calcium-sulfate, organic polymers, collagen, gelatin, polyether-etherketone (PEEK), ultra high molecular weight polyethylene (UHMWPE or UHMW), or combinations thereof.

Alternatively, implants can include or essentially consist of materials of autographic origin, materials of allogenic origin, materials of xenogenic origin or composites or mixtures of synthetic (metals or ceramics) and autographic, allogenic or xenogenic materials. Materials obtained or derived from autograft, allograft, or xenograft tissue are distinct from in vivo tissue in that the materials are processed to be suitable for implantation in humans. In particular embodiments, the implantation material is passivated material. As used herein, the term “passivate” is intended to refer to the elimination of potentially pathogenic organisms and immunogenic substances from an implant. Thus, both sterility and reduced antigenicity is intended by this term, although elimination of beneficial biological properties of the implant, such as osteogenic properties (osteoconduction or osteoinduction; bone fusion), natural tissue functionality, and desirable structural strength of an implant are not intended by this term. The term “passivation” is preferred to the term “sterilize” because, while sterilization is a goal, that term has an absolute connotation for which the ability to definitively test is limited by the state of the art of making such measurements and/or by the need for attendant tissue destruction. In addition, while the implants produced according to the method of this application may not be completely devoid of any antigenicity or pyrogenicity, these undesirable aspects are greatly reduced, and this too is intended by the term “passivation,” as used herein. Suitable processes for removing antigenic proteins and neutralizing any bacteria and viruses are known in the art. See, e.g., U.S. Pat. No. 5,846,484, U.S. Pat. No. 6,613,278, U.S. Pat. No. 6,482,584 and U.S. Pat. No. 6,652,818, all of which are incorporated herein by reference in their entirety.

Examples of implants of use in this invention include, but are not limited to, the AEGIS™ Anterior Lumbar Plate System, the BENGAL™ Stackable Cage System, the CHARITE® Artificial Disc, the CONCORDE™ Bullet System, the DISCOVERY® Screw System, the EAGLE™ Plus Anterior Cervical Plate System, the EXPEDIUM® 4.5 Spine System, the EXPEDIUM® 6.35 Spine System, the EXPEDIUM® PEEK Rod System, the EXPEDIUM® SFX™ Cross Connector System, the MONARCH® 5.50 Ti Spine System, the MOSS® MIAMI SI Spine System, the MOUNTAINEER™ OCT Spinal System, the SKYLINE™ Anterior Cervical Plate System, the SUMMIT™ SI OCT System, the UNIPLATE™ Anterior Cervical Plate System, the VIPER™ System, the VIPER™2 Minimally Invasive Pedicle Screw System and the X-MESH™ Expandable Cage System by DePuy Spine; the PINNACLE® Hip Solutions with TRUEGLIDE™ technology, the SIGMA®Knee products, the GLOBAL® Shoulder products, and the ANATOMIC LOCKED PLATING SYSTEMS (A.L.P.S.) by DePuy Orthopaedics; the replacement hip, knee, elbow, shoulder products as well as the spinal and trauma products by Zimmer; the replacement hip and knee products as well as the hand, spinal and trauma products by Stryker; the trauma products, intervertebral disks and fixation systems by Synthes; and the hip, knee, shoulder and finger prostheses by Mathys.

Dental implants are also included within the scope of this invention. Dental implants are introduced into the jaw in order to mount or fasten artificial teeth or prostheses. Examples of such implants include, but are not limited to, the SPI® products from Thommen Medical; the various implants including the NOBELACTIVE™ and NOBELREPLACE™ implants from Nobel Biocare; and the STRAUMANN® Bone Level Implants from Straumann.

The implant surface may be porous or non-porous and may be treated or have a roughened surface in order to improve the integration with the neighboring tissue (e.g., bone) and/or to speed up the healing process. Various methods for producing such surfaces are disclosed in the art.

The biomimetic graft or implant of the invention is produced by incubating a graft or implant with undifferentiated mesenchymal stem cells in a first culture medium for sufficient time to promote adhesion of the stem cells to the graft or implant; replacing the first culture medium with an osteogenic culture medium; incubating the graft or implant in the osteogenic culture medium to induce osteogenic differentiation of the human mesenchymal stem cells; and subjecting the graft or implant to decellularization to provide a graft or implant coated with an osteogenic extracellular matrix.

Mesenchymal stem cells (MSCs) are multipotent cells fundamentally characterized by their ability to differentiate into various mesenchymal tissues such as bone, cartilage, tendon, muscle and adipose tissue, among others. MSCs are present in different types of tissues such as bone marrow, limbal cells, adipose tissue, blood of the umbilical cord, etc. and constitute a population of cells that can be isolated and characterized by methods routinely practiced in the art (Pittenger & Martin (2004) Circ. Res. 95:9-20; Chan, et al. (2014) Cell Transplant. 23:399-406; Ding, et al. (2011) Cell Transplant. 20:5-14; Friedenstein, et al. (1976) Exp. Hematol. 45:267-274; Kastrinaki, et al. (2008) Tissue Eng. Part C Methods 14:333-339; Pendleton, et al. (2013) PLoS One 8:e58198; Thirumala, et al. (2009) Organogenesis 5:143-154). In certain embodiments of this invention, the MSCs are human MSCs (hMSCs). The Mesenchymal and Tissue Stem Cell Committee of the International Society for Cellular Therapy defines undifferentiated hMSC populations as including the following: (i) hMSCs must be plastic-adherent when maintained in classical culture conditions; (ii) hMSCs must express high levels (≧95% positive) of CD105, CD73, and CD90 and lack expression (≦2% positive) of CD45, CD34, CD14, or CD11b, CD79α or CD19, and HLA-DR (unless stimulated by interferon-γ) surface molecules; and (iii) hMSCs must differentiate into osteoblasts, adipocytes, and chondroblasts under specific in vitro differentiation conditions (Dominici, et al. (2006) Cytotherapy 8:315-317).

In accordance with the method herein, isolated MSCs are incubated with a graft or implant in a first culture medium for sufficient time to promote adhesion of the stem cells to the graft or implant. As used herein, the term “culture medium” relates to a liquid or solid nutrient preparation for the culturing, growth and/or proliferation of cells. In this respect, the first culture medium is a chemically defined medium, which promotes cell growth and attachment. In general, the first medium is a basal medium containing standard inorganic salts, vitamins, glucose, a buffer system and essential amino acids, wherein the basal medium is amended with different supplements (e.g., 10-20% serum or other defined factors) that promote the cell growth and attachment of MSCs, in particular hMSCs. Basal media include, e.g., Minimum Essential Medium (MEM); Dulbecco's Modified Eagle Medium (DMEM); Ham's F10 or F12 medium; MCDB 131, a medium developed by Knedler and Ham as a medium with reduced serum supplement for the growth of human cells; or Roswell Park Memorial Institute (RPMI) 1640. The formulation and composition of these media is widely known and can be obtained from any producer or supplier, such as for example Gibco (Life Technologies) or Sigma. By way of illustration, minimum essential medium-a (αMEM) supplemented with fetal bovine serum was shown herein to support the growth and adhesion of isolated hMSCs. The composition of αMEM is presented in Table 1.

TABLE 1

| | |
|---|---|
| Component | g/L |

Inorganic Salts

| | |
|---|---|
| CaCl₂•2H₂O | 0.2 |
| MgSO₄ (anhydrous) | 0.09767 |
| KCl | 0.4 |
| NaHCO₃ | 2.2 |
| NaCl | 6.8 |
| Na₂HPO₄ (anhydrous) | 0.122 |

Amino Acids

| | |
|---|---|
| L-Alanine | 0.025 |
| L-Arginine•HCl | 0.126 |
| L-Asparagine•H₂O | 0.05 |
| L-Aspartic Acid | 0.03 |
| L-Cysteine•HCl•H₂O | 0.1 |
| L-Cystine•2HCl | 0.0313 |
| L-Glutamic Acid | 0.075 |
| L-Glutamine | 0.292 |
| Glycine | 0.05 |
| L-Histidine•HCl•H₂O | 0.042 |
| L-Isoleucine | 0.052 |
| L-Lysine•HCl | 0.0725 |
| L-Methionine | 0.015 |
| L-Phenylalanine | 0.032 |
| L-Proline | 0.04 |
| L-Serine | 0.025 |
| L-Threonine | 0.048 |
| L-Tryptophan | 0.01 |
| L-Tyrosine•2Na•2H₂O | 0.0519 |
| L-Valine | 0.046 |

Vitamins

| | |
|---|---|
| L-Ascorbic Acid•Na | 0.05 |
| D-Biotin | 0.0001 |
| Choline Chloride | 0.001 |
| Folic Acid | 0.001 |
| Myo-Inositol | 0.002 |
| Niacinamide | 0.001 |
| D-Panthothenic Acid•1/2Ca | 0.001 |
| Pyridoxal•HCl | 0.001 |
| Riboflavin | 0.0001 |
| Thiamine•HCl | 0.001 |
| Vitamin B₁₂ | 0.00136 |

Other

| | |
|---|---|
| Adenosine | 0.01 |
| Cytidine | 0.01 |
| 2′-Deoxyadenosine | 0.01 |
| 2′-Deoxycytidine•HCl | 0.011 |
| 2′-Deoxyguanosine | 0.01 |
| Glucose | 1.0 |
| Phenol Red•Na | 0.011 |
| Pyruvic Acid | 0.11 |
| Thioctic Acid | 0.0002 |
| Thymidine | 0.01 |
| Uridine | 0.01 |

In certain embodiments, the first culture medium is a serum-free medium. Examples of a serum-free medium for promoting adhesion of MSCs are well-known in the art and commercially available. For example, CORNING STEMGRO is a chemically defined medium that enables adhesion and expansion of hMSCs. Moreover, as an alternative to serum, leucocyte/platelet coat lysate (i.e., buffy coat) can be used to promote adherence (U.S. Pat. No. 8,835,175).

Additional medium supplements include, e.g., insulin (Cartwright & Shah (2002) Culture Media. Basic Cell Culture, 2nd edition, Davis (ed) Oxford University Press, NY); sodium selenite, which increases the antioxidant capacity of the cells and reduces cell damage (Ebert, et al. (2006) Stem Cells 24:1226); transferrin; ethanolamine, which encourages the construction of cell membranes; basic fibroblast growth factor (bFGF), which promotes significant cell expansion either alone or in synergy with transforming growth factor-beta 1 (TGF-β1) (Jung, et al. (2010) Cytotherapy 12:637-57); and/or ascorbic acid, hydrocortisone and/or fetuin, which are important growth and attachment factors (Jung, et al. (2010) Cytotherapy 12:637-57).

The time required to promote adhesion of the stem cells to the graft or implant can vary depending on the condition of the cells and/or the nature of the surface of the graft or implant. In general, the cells are contacted with the surfaced of the graft or implant for at least 4 hours, 6 hours, 8 hours, 10 hours, 12 hours, 14 hours, 16 hours, 18 hours, 20 hours, 22 hours, 24 hours or up to 48 hours. There are four major steps that precede proliferation of cells on biomaterials: protein adsorption, cell-substrate contact, attachment and adhesion/spreading (Vogler (1988) Biophys. J. 53:759-69; Anselme (2000) Biomaterials 21:667-81; Wilson, et al. (2005) Tissue Eng. 11:1-18). Protein adsorption is a complex process that occurs rapidly, on the order of seconds, and is affected by many factors such as charge density, as proteins tend to be negatively charged (Arima & Iwata (2007) Biomaterials 28:3074-82; Renner, et al. (2005) Langmuir 21:4571-77; Wang, et al. (2006) J. Biomed. Mater. Res. A 77:672-8). In the second stage, cells will come into contact with the surface as dictated by physicochemical forces such as van der Wals (attractive) and electrostatic (repulsive) interactions. As the cells pass through the secondary energy minimum (where these attractive and repulsive forces balance), cells begin to make physical contact with the surface. Specific adhesion interactions then bring the cell to within approximately 15-50 nm of the surface, the primary energy minimum. The adhesion phase occurs over a longer time frame and involves more specific adhesive interactions with extracellular matrix proteins, the cell membrane and cytoskeletal proteins (Wilson, et al. (2005) Tissue Eng. 11:1-18). Attached cells then typically take hours to slowly spread over the surface, and begin to produce their own matrix. It is in this stage that adhesion proteins are produced by cells leading to the production of focal complexes or adhesions. The steady-state adhesion plateau can be approximated by the classic thermodynamic DLVO theory of colloid stability of attractive forces and repulsive barriers, the Dupre equation (Vogler (1988) Biophys. J. 53:759-69), specific receptor-ligand bonds (Vitte, et al. (2004) Eur. Cell. Mater. 7:52-63) or approximated by tₘₐₓ (i.e., the half-way point between the initiation of exponential growth and completion of attachment on the adhesion plateau)(Vogler (1988) Biophys. J. 53:759-69). The time required to promote adhesion of the stem cells to the graft or implant can also be assessed experimentally by washing the graft or implant and determining how many cells have adhered to the surface.

Once the MSCs have adhered to the graft or implant, the first culture medium is replaced with an osteogenic culture medium. An “osteogenic culture medium” is a medium that induces or stimulates the differentiation of MSCs, in particular hMSCs, into osteoblasts. The osteogenic culture medium is composed of a basal medium amended with one or more agents, growth factors or external stimulants to induce or stimulate osteogenesis. Such supplements include, but are not limited to, dexamethasone, ascorbic acid or L-ascorbic acid-2-phosphate and β-glycerol phosphate. See, e.g., Jaiswal, et al. (1997) J. Cell. Biochem. 64:295-312. Alternatively, the osteogenic culture medium can be obtained from a commercial source, e.g., ORICELL™ Mesenchymal Stem Cell Osteogenic Differentiation Medium.

The graft or implant and MSCs are subsequently incubated or cultured in the osteogenic culture medium to induce osteogenic differentiation of the MSCs. This step can be performed for at least 4, 5, 6, 7, 8, 10, 12, 15, 20, 25, 30, 35, or 40 days under standard culture conditions (e.g., 37° C., 5% CO₂). Preferably, this step is performed for between one and two weeks. Osteogenic differentiation can be determined by osteoblastic morphology, expression of alkaline phosphatase (ALP), reactivity with anti-osteogenic cell surface monoclonal antibodies, modulation of osteocalcin mRNA production, and/or the formation of a mineralized extracellular matrix containing hydroxyapatite as described herein.

Upon osteogenic differentiation, the graft or implant is subjected to decellularization to provide a graft or implant coated with an osteogenic extracellular matrix. The decellularization process of the present invention has the advantageous effect of removing cellular material while preserving the extracellular matrix proteins on the surface of the graft or implant thereby allowing the graft or implant to more easily and efficiently accept new cells, be surgically transplanted, and lead to a successful graft or implant in the recipient's body. Additionally, the decellularization process of the present invention has the effect of leaving the graft or implant relatively free from residual material left by the chemicals which contact human tissue. This allows for a cleaner, safer, and more efficient graft or implant procedure. Decellularization of the graft or implant can be achieved as exemplified herein or using other methods or combinations of methods including osmotic shock sequences, a detergent wash, an enzyme treatment, a RNA-DNA extraction, and/or an organic solvent extraction.

Osmotic shock sequences can include, e.g., contacting the graft or implant with a hypotonic solution (e.g., double deionized water, ddH₂O)), followed by a treatment with a hypertonic salt solution, followed by a second treatment with a hypotonic solution, preferably ddH₂O. A hypertonic salt solution can include normal saline, one or more chlorides, a sugar or sugar alcohol, and combinations thereof. Preferred chlorides include NaCl (0.9% to 3.0% (w/v)), MgCl₂ (1.0 to 5.0 mM), KCl (200 to 800 mM), and combinations thereof. Various sugars or sugar alcohols including mannitol (5% to 20% (w/v)), polysaccharides, polyols, dulcitol, rhamnitol, inositol, xylitol, sorbitol, rhamnose, lactose, glucose, galactose, and combinations thereof. Advantageously, these compositions dehydrate the tissue and prepare it for subsequent conditioning where the tissue is capable of more readily taking up or absorbing solutions in which the tissue is placed.

A detergent wash can include the use of one or more detergents such as nonionic, anionic, zwitterionic, detergents and combinations thereof. Nonionic detergents include, but are not limited to, chenodeoxycholic acid, chenodeoxycholic acid sodium salt, cholic acid, deoxycholic acid, deoxycholic acid methyl ester, digitonin, digitoxigenin, n,n-dimethyldodecylamine n-oxide, docusate sodium salt, glycochenodeoxycholic acid sodium salt, glycocholic acid hydrate, glycocholic acid sodium salt hydrate, glycocholic acid sodium salt, glycolithocholic acid 3-sulfate disodium salt, glycolithocholic acid ethyl ester, n-laurolysarcosine sodium salt, lithium dodecyl sulfate, lugol solution, NIAPROOF 4, TRITON, TRITON QS-15, TRITON QS-44 solution, TRIZMA dodecyl sulfate, Ursodeoxycholic acid, and combinations thereof. Examples of anionic detergents for use in the present invention, include, but are not limited to, BIGCHAP, Bis(polyethylene glycol bis[imidazoyl carbonyl]), BRIJ detergents, CREMOPHOR EL (Sigma, Aldrich), N-Decanoyl-N-methylglucamine, Polyethylene glycol ether, Polyoxyethylene, Saponin, SPAN detergents (Sigma Aldrich), Tergitol, Tetradecyl-b-D-maltoside, TRITON CF-21, TRITON X-100, TRITON X-15, TWEEN (Sigma Aldrich), Tyloxapol, and combinations thereof. Zwitterionic detergents include, but are not limited to, CHAPS, CHAPSO, Sulfobetaine 3-10 (SB 3-10), Sulfobetaine 3-(SB 3-12), Sulfobetaine 3-14 (SB 3-14), ZWITTERGENT detergents, and combinations thereof. In certain embodiments, the detergents used are TRITON X®-100 (TRITON), N-lauroylsarcosine Sodium Salt Solution (NLS), and combinations thereof. Preferably, the detergent wash has the effect of solubilizing proteins, lysing cells, and also acting as an anti-calcification agent. Generally, the detergent(s) is present in an amount of about 0.01% to 1% by volume.

Enzyme treatment for decellularization can include the use of one or more collagenases, one or more dispases, one or more DNases, or a protease such as trypsin. An exemplary enzyme of use in decellularization step is BENZONASE endonuclease, which removes DNA.

Organic solvent extraction typically includes the use of an alcohol such as ethyl alcohol, methyl alcohol, n-propyl alcohol, iso-propyl alcohol, n-butyl alcohol, sec-butyl alcohol, t-butyl alcohol, iso-amyl alcohol, n-decyl alcohol and combinations thereof. In some embodiments, the alcohol has a high concentration, preferably from about 20 proof to 70 proof. In certain forms, the alcohol also acts an anti-calcification agent, one such preferred alcohol is ethyl alcohol. In another embodiment, the extraction has the effect of sterilizing and disinfecting the graft or implant, as well as removing lipids and other hydrophilic residuals.

Advantageously, the decellularized graft or implant provides a biological scaffold, which includes materials and pore sizes that are biologically appropriate for recellularization and osseointegration and/or osteoinduction of the graft or implant. “Osseointegration” is used herein to refer to both osseointegration and osteointegration. Typically the term “osseointegration” is used when used in the dental field and “osteointegration” is used when used in the spinal/long bone field as well as when referring to integration of replacement joints (such as, e.g., hip, knee, shoulder, spine). However, both terms refer to the integration of the implant into the surrounding bone tissue.

As indicated herein, the decellularized graft or tissue includes a biomimetically or biologically appropriate extracellular matrix (ECM) component. ECM components can include any or all of the following: fibronectin, fibrillin, laminin, elastin, members of the collagen family (e.g., collagen I, III, and IV), glycosaminoglycans, ground substance, reticular fibers and thrombospondin, which can remain organized as defined structures such as the basal lamina. Successful decellularization is defined as the absence of detectable myofilaments, endothelial cells, smooth muscle cells, and nuclei. Preferably, but not necessarily, residual cell debris also has been removed from the decellularized graft or implant. Upon decellularization, the graft or implant coated with an osteogenic extracellular matrix may then be packaged and stored for use in surgical procedures.

The graft or implant resulting from the instant method is defined herein as “a biomimetic graft or implant coated with an osteogenic extracellular matrix,” which includes physiologically relevant amounts of growth factors, hydroxyapatite nucleating proteins and other structural proteins. In this respect, the term “coated” is used herein to mean that the extracellular matrix adheres to at least a portion of the surface of the graft or implant.

The graft or implant prepared in accordance with the method of the invention facilitates or promotes in vivo osteoinduction and/or osseointegration and is therefore of use in the treatment of a number bone-related injuries and conditions. Accordingly, the present invention also provides a method for promoting in vivo osteoinduction or osseointegration of a graft or implant by implanting a graft or implant coated with an osteogenic extracellular matrix in a subject at a site wherein bone tissue and said graft or implant are maintained at least partially in contact for a time sufficient to permit enhanced bone tissue growth between said tissue and said graft or implant. The level of osteoinduction or osseointegration of an implant can be determined by one of several methods. For example, the bone mineral density around an implant site, the area of bone/implant contact, bone volume, the force required to remove an implant, resonant frequency analysis and the torque required to remove the implant are all indicators of the level of osteoinduction and/or osseointegration.

Various methods for measuring bone mineral density are known in the art and include X-ray radiographs, Dual energy X-ray absorptiometry (DEXA), peripheral Dual energy X-ray absorptiometry (P-DEXA), dual photon absorptiometry (DPA), ultrasound, quantitative computed tomography (QCT), and Roentgen Stereophotogrammetry Analysis (RSA), which can be used to study implant micromotion using implants with tantalum beads as “landmarks”. Improved osteoinduction or osseointegration is said to be seen when the bone mineral density around the implant site is increased compared to a control implant which is not coated with an osteogenic extracellular matrix.

In one embodiment, the subject being treated has a fracture to a limb (i.e., leg or arm) or joint (e.g., knee or hip). Thus, the subject being treated has a fracture to one or more of the humerus, skull, pelvis, radius, ulnar, a carpal, a metacarpal, the clavical, scapular, femur, os coxae, patella, tibia, fibula, talus, calcaneus, a tarsal, a metatarsal, the ischium or the ileum. In another embodiment, the subject being treated has undergone, or will undergo surgery on one or more of the following joints: knee, hip, ankle, shoulder, elbow. Such surgery includes hip replacement and knee replacement. In one embodiment, the subject has a spinal injury or deformation due to illness or genetic disease. In certain embodiments, the subject is one who requires spinal fusion surgery.

In another embodiment, the subject being treated has received or will receive a dental implant.

In a further embodiment, the subject being treated is one who has been identified as having or as being at risk of suffering from osteoporosis. In one embodiment, the subject has a bone metabolic disease leading to low bone mass (BM) development and/or fractures. In another embodiment, the subject being treated is one who has osteogenesis imperfecta or hypophosphatasia. These embodiments include both (i) subjects at risk of fractures, and (ii) subjects not at risk of fractures. Such a subject may be identified by looking at, for example, nutritional intake, family history, genetic markers, medical examination, serological bone biomarkers, and bone mineral density by DEXA, and overall fracture assessment by FRAX™.

Subjects that can be treated in accordance with the method of the invention include mammals such as humans who are less than 5 years old, 5-10 years old, 10-20 years old, 20-30 years old, or 30-40 years old. In one embodiment, the subject is 40 years of age or older, 50 years of age or older, 60 years of age or older, or 70 years of age or older. In one embodiment, the subject is a post-menopausal woman.

This invention also provides a kit containing a biomimetic graft or implant coated with an osteogenic extracellular matrix for use in promoting in vivo osteoinduction and/or osseointegration. In accordance with the kit, the graft or implant is preferably passivated and provided in a lyophilized form in a sterile package. In certain embodiments, the kit includes the graft or implant and instructions for use. In some embodiments, the graft or implant is a surgical implant. In other embodiments, the graft or implant is a dental implant.

The following non-limiting examples are provided to further illustrate the present invention.

### EXAMPLE 1

### Osteogenic/Osteointegrating ECM Coating on Titanium Implants

Dental titanium implants were placed in a 96-well cell culture dish, base down, and incubated for 16 hours with a human mesenchymal stem cell (HMSC) suspension (200 til containing 500,000 cells) at 37° C., 5% CO₂ in standard cell culture media (αMEM with 20% FBS and 1% antibiotic/antimycotic solution). Subsequently, the implants were transferred aseptically into 24-well cell culture plates with one implant placed in each well. The implants were incubated for a further 24 hours in standard cell culture media to facilitate proper cell attachment to the implants. After 24 hours of culture, the media was changed to an osteogenic culture medium to trigger osteogenic differentiation of the HMSCs. The osteogenic culture medium was made using the standard culture medium amended with dexamethasone (10 mM), ascorbic acid (100 μg/ml) and β-glycerophosphate (10 mM). The implants were cultured in this media for a period of 2 weeks with the media changed every other day. After 2 weeks, the implants were decellularized using the following procedure:

1. The cell culture media was removed and the implants were incubated with Buffer 1 (10 mM sodium phosphate, 150 mM sodium chloride, and 0.5% TRITON X-100) for 1 hour at 37° C., 5% CO₂.

2. Buffer 1 was removed and the implants were incubated in Buffer 2 (25 mM ammonium hydroxide) for 1 hour at 37° C., 5% CO₂.

3. Buffer 2 was removed and the implants were washed three times in Hank's Balanced Salt Solution (HBSS).

4. The wash buffer was removed and the implants were incubated with a solution of DNAse containing 50 units of DNAse per implant in a volume of 1 ml for 1 hour at 37° C.

5. The DNAse solution was removed and the implants were washed three times in HBSS solution followed by 3 times washing in double deionized water.

6. The excess water from the implants was blotted and the implants were frozen overnight at −80° C. followed by incubation for 24 hours in a lyophilizer.

7. The implants were removed from the lyophilizer and stored aseptically at room temperature.

### EXAMPLE 2

### Biomimetically Enhanced Demineralized Bone Matrix (DBM)

DBM granules used for clinical bone regeneration were placed inside a 24-well cell culture plate. HMSCs were seeded onto the DBM at a concentration of 1 million cells per 250 mg of DBM granules and cultured in standard HMSC culture media for a period of 24 hours. Subsequently, the media was changed to osteogenic culture medium and the cells were cultured for a period of 2 weeks with media changed every other day. This was performed to induce osteogenic differentiation of the HMSCs and to facilitate the generation of a pro-osteogenic matrix. After 2 weeks, the DBM granules containing HMSCs were decellularized using the following procedure:

1. The cell culture medium was aspirated and the granules were incubated in Buffer 1 (see Example 1) for 1 hour.

2. Buffer 1 was removed and the granules were incubated in Buffer 2 (see Example 1) for 2 hours.

3. Buffer 2 was removed and the granules were washed three times in HBSS.

4. HBSS was removed and the granules were incubated with DNAse solution (100 units of DNAse per 250 mg of granules) for 1 hour.

5. The DNAse solution was removed and the granules were washed three times in HESS followed by three washes in double deionized water.

6. The granules were then frozen at −80° C. overnight and lyophilized for 24 hours.

7. The lyophilized biomimetically enhanced DBM (EDBM) granules were then stored aseptically at room temperature.

### EXAMPLE 3

### Assessing Osteogenic Differentiation

Alkaline Phosphatase Activity and Mineralization. The osteogenic differentiation capacity of hMSCs can be determined at 7 and 14 days by analyzing ALP activity and mineralization. ALP is a generally used marker for early osteogenic differentiation, whereas mineralization of the ECM is a characteristic of late osteogenic differentiation. Quantitative ALP analysis can be performed using an ALP Kit (Sigma-Aldrich) according to established methods (Tirkkonen, et al. (2011) J. R. Soc. Interface 8:1736-47). A quantitative Alizarin Red S method can be used at 7 and days to detect mineralization. See, Tirkkonen, et al. (2011) J. R. Soc. Interface 8:1736-47. Briefly, ethanol fixed cells are stained with 2% Alizarin Red S solution (Sigma-Aldrich), and photographed after several steps of washing. Cetylpyridinium chloride (Sigma-Aldrich) is used to extract the dye, followed by absorbance measurement at 540 nm with a microplate reader.

Quantitative Real-Time PCR. Quantitative real-time reverse transcription polymerase chain reaction (qRT-PCR) analysis is used to compare the relative expression of osteogenic genes under different culturing conditions. For qRT-PCR analysis, hMSCs are seeded on 6-well plates at a density of 7×10³ cells/cm². A CELLstart pre-coating of well plates is used in xeno-free conditions. Total RNA is isolated from the cells at 7- and 14-day time points with Nucleospin kit reagent (Macherey-Nagel GmbH & Co. KG, Düren, Germany) according to manufacturer's instructions. First-strand cDNA is synthesized from total RNA using the High-Capacity cDNA Reverse Transcriptase Kit (Applied Biosystems, Foster City, Calif.). The expression of osteogenic genes including runx2A, DLX5, collagen type I, osteocalcin, and ALP is analyzed. Isoform A of runx2 is analyzed due to its specificity for osteogenic differentiation in comparison to isoform C (Komori (2010) Cell tissue Res. 339:189-95; Banerjee, et al. (2001) Endocrinology 142:4026-39). Data is normalized to the expression of RPLPO (human acidic ribosomal phosphoprotein P0), a housekeeping gene, which has stable expression under different experimental conditions in similar studies (Gabrielsson, et al. (2005) Obes. Res. 13:649-52; Fink, et al. (2008) BMC Mol. Bio. 9:98). The qRT-PCR mixture contains 50 ng cDNA, 300 nM forward and reverse primers, and SYBR Green PCR Master Mix (Applied Biosystems). The reactions are conducted with, e.g., an ABIPRISM 7000 Sequence Detection System (Applied Biosystems) with initial enzyme activation at 95° C. for 10 minutes, followed by 45 cycles of denaturation at 95° C. for 15 seconds and anneal and extend at 60° C. for 60 seconds. The expression levels of all differentiation cultures are compared to the expression level of FBS control cultures.

What is claimed is:

1. A biomimetic graft or implant coated with an osteogenic extracellular matrix.

2. The biomimetic graft or implant of claim 1, wherein the graft or implant is a surgical implant or dental implant.

3. The biomimetic graft or implant of claim 1, wherein the graft or implant comprises titanium, ceramic or demineralized bone matrix.

4. A kit comprising a biomimetic graft or implant coated with an osteogenic extracellular matrix.

5. A method for producing a biomimetic graft or implant comprising
(a) incubating a graft or implant with undifferentiated mesenchymal stem cells in a first culture medium for sufficient time to promote adhesion of the mesenchymal stem cells to the graft or implant;
(b) replacing the first culture medium with an osteogenic culture medium;
(c) incubating the graft or implant in the osteogenic culture medium to induce osteogenic differentiation of the mesenchymal stem cells; and
(d) subjecting the graft or implant to decellularization thereby producing a biomimetic graft or implant coated with an osteogenic extracellular matrix

6. The method of claim 5, wherein the graft or implant is a surgical implant or dental implant.

7. The method of claim 5, wherein the graft or implant comprises titanium, ceramic or demineralized bone matrix.

8. A biomimetic graft or implant produced by the method of claim 5.

9. A method for promoting in vivo osseointegration or osteoinduction of a graft or implant comprising implanting a biomimetic graft or implant coated with an osteogenic extracellular matrix in a subject at a site wherein bone tissue and the biomimetic graft or implant are maintained at least partially in contact for a time sufficient to permit enhanced bone tissue growth between the tissue at the site and the biomimetic graft or implant.
